(19) United States
(12) Patent Application Publication
(10) Pub. No.: US 2026/0199131 A1
(43) Pub. Date: Jul. 16, 2026

(54) A GLAUCOMA DEVICE

(71) Applicant: VIVID WHITE PTY LTD, Melbourne VIC (AU)

(72) Inventors: Michael COOTE, Fitzroy (AU); Andrew BATTY, Beaumaris (AU); Craig Ross, Park Orchards (AU); Christopher Smith, Carnegie (AU)

(73) Assignee: VIVID WHITE PTY LTD, Melbourne VIC (AU)

(21) Appl. No.: 19/138,011

(22) PCT Filed: Dec. 22, 2023
(86) PCT No.: PCT/AU2023/051373

§ 371 Date: Jun. 11, 2025

(30) Foreign Application Priority Data
Dec. 28, 2022 (AU) 2022904037

(51) Int. Cl. A61F 9/007

(52) U.S. Cl. CPC A61F 9/00781; A61F 2250/0067

(57) ABSTRACT

There is disclosed a glaucoma device (10, 1010) comprising: an implant body (15, 1015) including: a frontal portion (20, 1020); a distal portion (35, 1035); a plurality of arm portions (40, 1040) extending between the frontal portion (20, 1020); and the distal portion (35, 1035) so as to provide a connection therebetween; and a fluid network extending from the frontal portion(20, 1020), along at least one of the arm portions (40, 1040), and to the distal portion (35, 1035), wherein the fluid network is provided with a plurality of exit channels (90, 1090) for egress of fluid therefrom; and a fluid entry cannula (30, 1030) disposed at the frontal portion (20, 1020) of the implant body (15, 1015) and adapted for fluid connection with ocular tissue.

### CROSS-REFERENCE TO RELATED APPLICATION

This application claims the benefit of priority to Australian Provisional Patent Application No. 2022904037, filed on 28 Dec. 2022. The entire disclosure of Australian Patent Application No. 2022904037 is hereby incorporated by reference in its entirety and for all purposes.

### FIELD

The present invention relates to a glaucoma device and components thereof. In particular, the invention relates to a device and components thereof for reducing intra-ocular pressure in an eye.

### BACKGROUND

Glaucoma is a progressive optic neuropathy whose principal risk factor is elevated Intra-Ocular Pressure (IOP). Untreated glaucoma causes irreversible loss of vision and is the most common cause of irreversible blindness worldwide.

As glaucoma is a chronic disease, it requires ongoing treatment and management to delay its progression. Existing management strategies for glaucoma rely on a reduction in the IOP of the eye, which may occur through topical medication, laser, or surgery. Current glaucoma surgical techniques have a relatively high level of variability and failure, and are costly to manage due to high post-operative demands.

Glaucoma surgery failure has been attributed to wound healing, and all efforts to date have focused upon anti-scarring strategies. In spite of this, glaucoma operations continue to create complications and fail. Work performed over a number of years has proven that fluid flow alone can cause failure of the operation - a significant issue as fluid flow is central to the effectiveness of the operation.

### SUMMARY

It is an object of the present invention to substantially overcome, or at least ameliorate, one or more of the disadvantages of existing arrangements, or at least provide a useful alternative to existing arrangements.

There is disclosed herein a glaucoma device comprising:
an implant body including:
   a frontal portion;
   a distal portion;
   a plurality of arm portions extending between the frontal portion and the distal portion so as to provide a connection therebetween; and
   a fluid network extending from the frontal portion, along at least one of the arm portions, and to the distal portion, wherein the fluid network is provided with a plurality of exit channels for egress of fluid therefrom; and
a fluid entry cannula disposed at the frontal portion of the implant body and adapted for fluid connection with ocular tissue.

The fluid network may include:
a fluid entry channel provided in the frontal portion;
a fluid carrier channel provided in at least one of the arm portions to carry fluid distally; and
a distal fluid distributor channel provided in the distal portion and connected to the plurality of exit channels.

The fluid network may further include an anterior fluid distributor channel in at least one

of the arm portions to carry fluid anteriorly to the plurality of exit channels.

The fluid network may further include:
a fluid distributor hub providing a fluid connection between the fluid carrier channels and the distal fluid distributor channel, and wherein the anterior fluid distributor channels extend from the fluid distributor hub.

The fluid network may extend from the fluid entry channel provided in the frontal portion, along each of the fluid carrier channels of the arm portions, towards the distal fluid distributor channel provided in the distal portion, and returning along each of the anterior fluid distributor channels of the arm portions.

The plurality of exit channels may be provided in each of the distal and anterior fluid distributor channels so as to allow for egress of fluid from one or more of the arm portions preferentially in the distal portion.

The fluid network may be provided with between approximately 70 and 220 of the exit channels. In one form, the fluid network may be provided with approximately 190 of the exit channels.

The fluid network may alternatively be provided with between approximately 150 and 180 of the exit channels. In one form, the fluid network may be provided with approximately 163 of the exit channels.

The fluid network may be adapted to provide a resistance to aqueous humour fluid flow of between 2.00 to 4.80 mmHg·min/uL (Hagen-Poiseuille flow with fluid dynamic viscosity of 0.72 mPa·s). In one form, the fluid network may be adapted to provide a resistance to aqueous humour fluid flow of between 3.00 to 3.80 mmHg·min/uL. In another form, the fluid network may be adapted to provide a resistance to aqueous humour fluid flow of between 2.80 to 3.80 mmHg·min/uL. The resistance may be measured with or without the use of flow modification such as an intraluminal stent.

The entry cannula may include:
a first end portion having a first opening adapted for connection with the ocular tissue; and
a second end portion having a second opening connected to a fluid port of the frontal portion of the implant body; and
a lumen extending between the first and second openings to provide for fluid connection between the first and second end portions.

The entry cannula may extend at an angle of approximately 145 degrees relative to the implant body. In another form, the entry cannula may extend at an angle of approximately 150 degrees relative to the implant body

The entry cannula may be articulated separately from the implant body.

The plurality of arm portions may include:
an inner arm portion;
a first lateral arm portion disposed on a first side of the inner arm portion and spaced therefrom to form a first window; and
a second lateral arm portion disposed on a second side of the inner arm portion and spaced therefrom to form a second window.

The distal portion may extend between the first lateral portion to the second lateral portion so as to form a third window between the plurality of arm portions and the distal portion.

The implant body may have a length of between approximately 16 and 23 millimetres and a width of between approximately 5 and 8 millimetres.

The implant body may have a length of approximately 20 millimetres and a width of approximately 6.5 millimetres. In another form, the implant body may have a length of approximately 19.5 millimetres and a width of approximately 5.5 millimetres.

The implant body may have a thickness of less than approximately 0.6 millimetres.

The implant body may have a thickness of between approximately 0.15 millimetres to 0.6 millimetres. In another form, the implant body may have a thickness of approximately 0.21 millimetres.

The implant body may have a thickness that gradually tapers from the frontal portion to the distal portion.

The implant body may have a thickness of approximately 0.2 millimetres at the frontal portion and approximately 0.4 millimetres at the distal portion.

The implant body may further include at least one suture hole for surgical fixation of the implant body to the ocular tissue.

The at least one suture hole may be provided adjacent the plurality of arm portions. The plurality of exit channels may be provided in a fluid distributor channel surrounding the suture hole so as to allow for egress of fluid thereform.

Each of the arm portions may have a width of approximately 1 millimetre and a thickness or depth that gradually tapers from 0.25 to 0.4 millimetres.

The implant body may be formed from a flexible or elastomeric polymer material.

The implant body may be formed from a medical grade Polydimethylsiloxane (silicone).

There is further disclosed herein a glaucoma device comprising:
an implant body including:
   a frontal portion adapted for fluid connection with ocular tissue;
   a distal portion;
   a plurality of arm portions extending between the frontal portion and the distal portion so as to provide a connection therebetween; and
   a fluid network to provide internal fluid resistance, whereby the fluid network extends from the frontal portion, along at least one of the arm portions, and to the distal portion, wherein the fluid network is provided with a plurality of exit channels for egress of fluid therefrom.

There is further disclosed herein a glaucoma device comprising:
an implant body including:
   a frontal portion;
   a distal portion;
   an arm portion extending between the frontal portion and the distal portion so as to provide a connection therebetween; and
   a fluid network extending from the frontal portion, along at the arm portion, and to the distal portion, wherein the fluid network is provided with a plurality of exit channels for egress of fluid therefrom; and
   an entry cannula disposed at the frontal portion of the implant body and adapted for fluid connection with ocular tissue.

There is further disclosed herein an entry cannula for a glaucoma device, the entry cannula having a cannula body including:
a first end portion having a first opening adapted for connection with ocular tissue; and
a second end portion having a second opening adapted for connection with an implant body of a glaucoma device; and
a lumen extending between the first and second openings to provide for fluid connection between the first and second end portions.

The second end portion of the cannula body may be wider than the first end portion of the cannula body.

The first end portion may have a bevelled or removed portion for the lumen to open anteriorly from the iris tissue.

The first end portion may have a rounded portion for ease of insertion into the ocular tissue.

The lumen may have a diameter of at least 0.08 millimetres.

The lumen may have a diameter of approximately 0.127 millimetres.

The cannula body may have an ellipse-shaped cross-section.

The cannula body may have a length of approximately 1.60 millimetres. In another form, the cannula body may have a length of approximately 2.00 millimetres.

The cannula body may have a width of between approximately 0.59 to 0.75 millimetres. In another form, the cannula body may have a width of between approximately 0.64 to 0.82 millimetres.

The cannula body may have a thickness or depth of between approximately 0.28 to 0.40 millimetres. In another form, the cannula body may have a thickness or depth of between approximately 0.31 to 0.43 millimetres.

The cannula body may further include one or more flanges that project outwardly from the cannula body, so as to enhance sealing properties of the cannula body.

Each flange may project outwardly from the cannula body in a direction parallel to the implant body of the glaucoma device and parallel to the corneal tissue.

Each flange may project outwardly from the cannula body at an angle of between approximately 145 and 155 degrees. In one form, each flange may project outwardly from the cannula body at an angle of approximately 150 degrees.

Each flange may have a flared configuration.

Each flange may have a width of approximately 0.75 millimetres and a depth/thickness of approximately 0.4 millimetres. In another form, each flange may have a width of approximately 0.815 millimetres and a depth/thickness of approximately 0.422 millimetres.

The one or more flanges may include:
a first flange disposed adjacent and spaced apart from the a surface of the second end portion of the cannula body;
a second flange spaced apart from the first flange; and
a third flange spaced apart from the second flange.

The first flange may be spaced apart from the surface of the second end portion by a distance of approximately 0.125 millimetres.

The second flange may be spaced apart from the first flange by a distance of approximately 0.125 millimetres, and the third flange is apart from the second flange by a distance of approximately 0.125 millimetres.

### BRIEF DESCRIPTION OF THE DRAWINGS

For a more complete understanding of the present invention, exemplary embodiments of the invention are explained in more detail in the following description with reference to the accompanying drawing figures, in which like reference signs designate like parts and in which:

FIG. 1 is a schematic isometric view of a glaucoma device with a fluid entry cannula according to one embodiment;

FIG. 2 is a schematic side view of the glaucoma device shown in FIG. 1, without the entry cannula;

FIG. 3 is a schematic plan view of the glaucoma device shown in FIG. 1, without the entry cannula;

FIG. 4 is a schematic bottom view of the glaucoma device shown in FIG. 1, without the entry cannula;

FIG. 5 is a schematic isometric view of an entry cannula according to one embodiment;

FIG. 6 is a schematic front view of the entry cannula shown in FIG. 5;

FIG. 7 is a schematic side view of the entry cannula shown in FIG. 5;

FIG. 8 is a schematic plan view of the entry cannula shown in FIG. 5

FIG. 9 is a schematic isometric view of a glaucoma device with a fluid entry cannula according to another embodiment;

FIG. 10 is a schematic side view of the glaucoma device shown in FIG. 9

FIG. 11 is a schematic plan view of the glaucoma device shown in FIG. 9; and

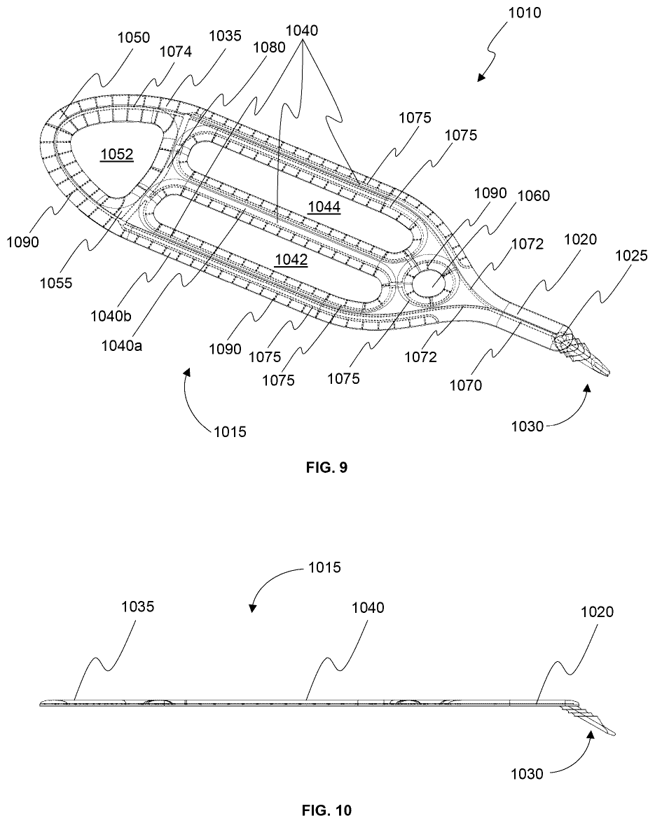

FIG. 10 is a schematic bottom view of the glaucoma device shown in FIG. 9.

### DETAILED DESCRIPTION

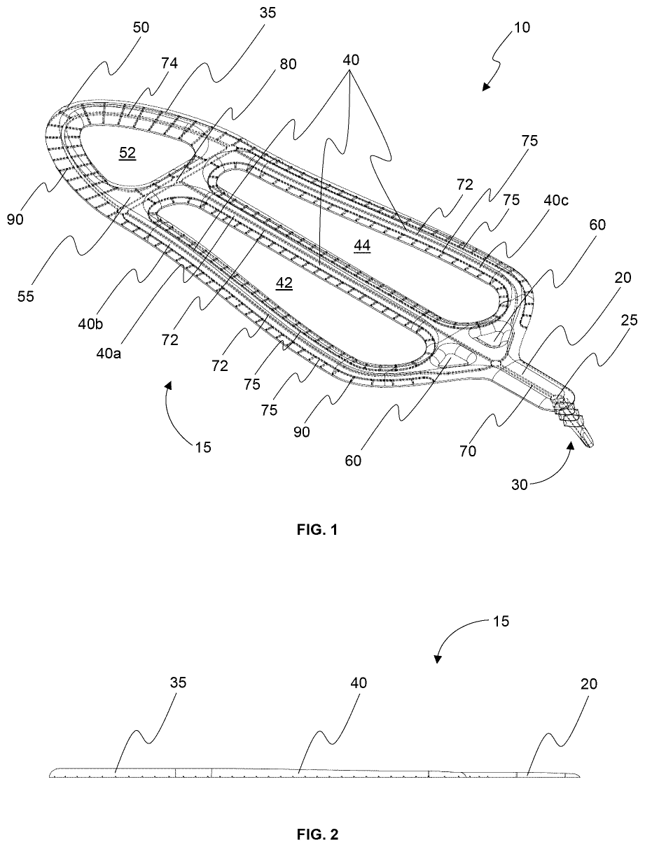

In FIGS. 1 to 8 of the accompanying drawings, there is schematically depicted an embodiment of a glaucoma device 10 and components thereof that are adapted for the treatment or management of Intra-Ocular Pressure (IOP) in the eye. The glaucoma device 10 is adapted to be surgically implanted into the sub-conjunctival space of an eye to drain and/or disperse aqueous humor.

The glaucoma device 10 includes an implant body 15 that is preferably formed from a flexible or elastomeric biocompatible material. A suitable biocompatible material may include, but is not limited to, a polymer material such as silicone or polyurethane. In one form, the material may be a medical grade Polydimethylsiloxane (silicone), or PDMS. It is envisaged that the overall dimensions of the implant body 15 may be between approximately 10 and 25 millimetres (mm) in length, less than approximately 10 mm in width, and less than approximately 0.5 mm in thickness/depth. In one form, the implant body may have a length of between approximately 16 and 23 mm, a width of between approximately 5 and 8 mm, and a thickness/depth of less than approximately 0.6 mm. In one preferred form, the implant body 15 has a length of approximately 20 mm, a width of approximately 6.5 mm, and a gradually tapering thickness/depth of between approximately 0.15 mm to 0.6 mm. In a further preferred form, the implant body 15 has a gradually tapering thickness/depth of between approximately 0.2 mm to 0.4 mm. It will, however, be appreciated that the dimensions of the implant body 15 are not necessarily limited to the preferred dimensions indicated above, and may be adjusted depending on the design requirements of the glaucoma device 10.

The implant body 15 includes a stem or frontal (anterior) portion 20 which may include a fluid port 25 therein. The fluid port 25 may provide an opening that allows the implant body 15 to be fluidly connected to ocular tissue. In one embodiment, and as will be described in further detail below, the glaucoma device 10 may further include or be provided with a fluid entry cannula 30 to facilitate the fluid connection between the implant body 15 and the ocular tissue, and in particular, the anterior chamber of the eye. It is understood that the implant body 15 is intended to lie against the sclera and between the rectus muscles of the eye.

In the depicted embodiment, and as best shown in FIG. 1, the frontal portion 20 is elongate and may have a width of between approximately 0.5 mm to 2 mm. In one form, the frontal portion 20 may have a length of approximately 2.5 mm, a width of approximately 1.3 mm, and a low-profile thickness/depth that gradually tapers from approximately 0.21 mm to 0.25 mm. As noted above in relation to the overall implant body 15 dimensions, it will be appreciated that the dimensions of the frontal portion 20 are not necessarily limited to the preferred dimensions indicated above, and may be adjusted depending on the design requirements of the glaucoma device 10. The arrangement of the frontal portion 20 may at least allow for surgical handleability, whereby the flat or low-profile surface (which may include smooth edges) may allow it to be pressed and fluidly sealed against ocular tissue (possibly with assistance from a surgical suture, as will be explained in further detail below). The low-profile, smooth edges, and gradually tapering thickness/depth of the frontal portion 20 may also at least reduce the risk of erosion through tissue.

The implant body 15 further includes a distal portion 35, and may also further include a plurality of arm portions 40 that extend between the frontal portion 20 and the distal portion 35. In the depicted embodiment, the implant body 15 includes three elongate arm portions, being an inner (central) arm portion 40a, a first lateral (peripheral) arm portion 40b, and a second lateral (peripheral) arm portion 40c. The inner arm portion 40a extends along a central longitudinal axis of the implant body 15. The first lateral arm portion 40b is disposed on a first side of the inner arm portion 40a and is spaced therefrom by a first opening or window 42. Mirroring the first lateral arm portion 40b is the second lateral arm portion 40c, which is disposed on a second side of the inner arm portion 40a and is spaced therefrom by a second opening or window 44. In one form, each of the arm portions 40 may have a width of 1 mm and a thickness/depth that that gradually tapers from 0.25 mm to 0.4 mm. The number of arm portions 40 provided may be adjusted depending on the design requirements of the glaucoma device 10. In certain embodiments (not shown), the implant body 15 may be provided with a single arm portion 40 that extends between the frontal portion 20 and the distal portion 35. It is understood, however, that increasing the number of arm portions 40 may in turn increase the number of exit channels (described in detail below) to reduce the velocity of fluid outflow so as to gently perfuse fluid and protect the tissue.

As will be appreciated in the depicted embodiment, the arm portions 40a, 40b, and 40c branch out symmetrically from the frontal portion 20, and may therefore increase the effective surface area of the implant body 15 and spread out the fluid egress locations, as will be described in further detail below. As discussed above, the implant body 15 may have a thickness/depth that gradually tapers from between approximately 0.2 mm (at the frontal portion) to approximately 0.4 mm (at the distal portion). This arrangement may at least aid with ease of implantation (rigidity) and may also increase the surface area of the implant body 15. It will, however, be appreciated that the implant body 15 may not necessarily include a tapering thickness/depth, and may have a constant thickness/depth, depending on the design requirements of the device 10.

The distal portion 35 preferably has a smooth or rounded form provided by an arcuate member 50 that may surround a third opening or window 52. In the embodiment whereby the implant body 15 has three arm portions 40 as depicted in the Figures, the arcuate member 50 extends or loops around between the first lateral arm portion 40b and the second lateral arm portion 40c. Accordingly, the third window 52 may be provided between the arcuate member 50 and the arm portions 40. The arcuate member 50 of the distal portion 35 may have a width of approximately 5.5 mm. The distal portion 35 is envisaged to be at least 20% less than the width of the overall implant body 15 (which, as indicated above, may be approximately 6.5 mm). This arrangement may at least aid with ease of implantation of the implant body 15.

A bridge portion 55 may be disposed between the arcuate member 50 of the distal portion 35 and the arm portions 40 to surround the third window 52. In the depicted embodiment, the bridge portion 55 also connects the arm portions 40a, 40b, and 40c. It will be appreciated that this arrangement of the distal portion 35 may at least increase the surface area and effective surface area of the implant body 15.

It will also be appreciated that the top edges of each portion of the implant body 15 (i.e. the frontal portion 20, the distal portion 35, and the arm portions 40) are preferably smooth or rounded to reduce risk of erosion into adjacent tissues. In one form, the top edges/corners may have a radius of at least 0.4 mm. It is also envisaged that the top surface area of the implant body 15 may be less than the footprint area (i.e. the area created by the footprint perimeter of the implant body 15). In one form, the top surface area of the implant body is less than 60% of the footprint area of the implant body 15. In this respect, the footprint area of the implant body 15 is envisaged to be at least 100 mm².

The implant body 15 may further include one or more suture openings or holes 60 for surgical fixation of the implant body 15 to the ocular tissue. In the depicted embodiment, the implant body 15 includes two suture holes 60 that are disposed between the plurality of arm portions 40 and the frontal portion 20. The suture holes 60 may at least provide a region for fixation of the implant body with a suture, such as a 10-0 Nylon suture. In the embodiment shown, the suture holes 60 may have a triangular shape, although it will be appreciated that the shape and configuration of the suture holes 60 are not necessarily limited to the shape and configuration shown, and may be adjusted depending on the design requirements of the glaucoma device 10. In other embodiments (not shown), the implant body 15 may be provided without suture openings or holes.

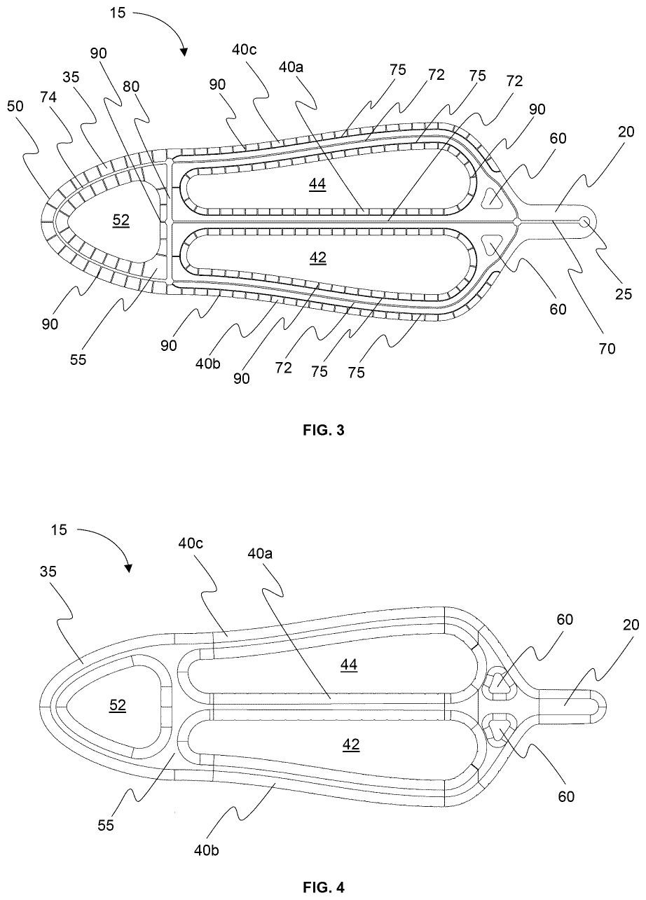

The implant body 15 further includes a fluid network that extends from the frontal portion 20, along at least one of the arm portions 40, to the distal portion 35. In the depicted embodiment, and as best shown in FIGS. 1 and 3, the fluid network commences at the fluid port 25 of the frontal portion 20 where fluid from the ocular tissue may enter the implant body 15, branches out in a first direction along each of the arm portions 40 towards the bridge portion 55, loops around the distal portion 35, and then returns in a second direction (opposite the first direction) along each of the arm portions 40. It will be appreciated that the fluid network may at least provide the implant body 15 with internal microfluidic resisting channels to manage tissue response.

The fluid network may include a fluid entry channel 70 provided in the frontal portion 20, a fluid carrier channel 72 provided in at least one of the arm portions 40, and a distal fluid distributor channel 74 provided in the distal portion 35. The fluid carrier channel 72 may have a width of approximately 0.051 mm and the fluid distributor channel 74 may have a width of approximately 0.035 mm. The fluid network may also include one or more anterior fluid distributor channels 75 provided in one or more of the arm portions 40. In the embodiment whereby the implant body 15 has three arm portions 40 as depicted in the Figures, each of the arm portions 40a, 40b, and 40c includes the fluid carrier channel 72 and at least one of the anterior fluid distributor channels 75. It will be understood that one or more of the fluid carrier channel 72, the distal fluid distributor channel 74, and the anterior fluid distributor channel 75 may at least allow for carriage of fluid to the various portions of the implant body 15, and to distribute fluid to various perfusion exit channels as will be described in further detail below. Each channel 70, 72, 74, and 75 may have internal dimensions of greater than 0.01 mm in width.

The fluid network may further include a fluid distributor hub 80 located in the bridge portion 55 and that provides a fluid connection between each of the fluid carrier channels 72 of the arm portions 40 and the distal fluid distributor channel 74. As best shown in FIGS. 1 and 3, fluid from the ocular tissue may travel along the fluid entry channel 70, along each of the fluid carrier channels 72, along the distal fluid distributor channel 74, and along the fluid distributor hub 80. The fluid may also loop back over the arm portions 40 via the anterior fluid distributor channels 75. In the embodiment shown, four anterior fluid distributor channels 75 are provided. A first anterior fluid distributor channel 75 extends from the fluid distributor hub 80 along a length of the lateral arm portion 40b. Likewise, a second anterior fluid distributor channel 75 extends from the fluid distributor hub 80 along a length of the lateral arm portion 40c. A third anterior fluid distributor channel 75 is provided as a loop that extends from the fluid distributor hub 80 and between the lateral arm portion 40b and the inner arm portion 40a (surrounding the window 42). Likewise, a fourth further fluid distributor channel 75 is provided as a loop that extends from the fluid distributor hub 80 and between the lateral arm portion 40c and the inner arm portion 40a (surrounding the window 44).

Accordingly, in the depicted embodiment, the fluid network extends from the fluid entry channel 70 provided in the frontal portion 20, along each of the fluid carrier channels 72 of the arm portions 40, towards the fluid distributor channel 74 provided in the distal portion 35, the fluid distributor hub 80, and the anterior fluid distributor channels 75 provided in the arm portions 40. The fluid network may have an overall length of greater than approximately 50 mm and an overall height of between 0.035 to 0.040 mm. In one form, the fluid network has an overall length of approximately 200 mm, and in a preferred form, the fluid network has an overall length of approximately 194 mm. It will, however, be appreciated that the length of the fluid network is not necessarily limited to the preferred length indicated above, and may be adjusted depending on the design requirements of the glaucoma device 10.

The fluid network may be provided with a plurality of exit channels 90 for egress of fluid therefrom (i.e. to the subconjunctival space) by way of perfusion. In the interest of clarity, not all of the exit channels 90 are accorded a reference numeral in the Figures. In the depicted embodiment, the exit channels 90 are provided with one or more of the fluid distributor hub 80, the distal fluid distributor channel 74 of the distal portion 35, and the anterior fluid distributor channels 75 of the arm portions 40. It will be appreciated that each exit channel 90 may extend in a direction transverse to the direction of extension of the respective hub 80 or channel 74, 75. In some embodiments (not shown), exit channels may be provided in one or more of the fluid carrier channels 72 or any other portion of the implant body 15. Accordingly, the exit channels 90 may allow for egress of fluid from one or more of the arm portions 40, the bridge portion 55, and/or the distal portion 35 of the implant body 15.

It is envisaged that the fluid network may include greater than 20 exit channels 90. In one form, the fluid network includes between 70 to 220 of the exit channels 90. In another form, the fluid network includes between 150 and 180 of the exit channels 90, and in a preferred form, the fluid network includes between 160 and 170 of the exit channels 90. In a further preferred form, the fluid network includes 163 of the exit channels 90. It will, however, be appreciated that the number of exit channels 90 provided are not necessarily limited to the preferred quantities indicated above, and may be adjusted depending on the design requirements of the glaucoma device 10. The exit channels 90 may be spaced apart from one another by up to 2 mm. In one form, the exit channels 90 are spaced apart from one another by between 0.30 to 0.80 mm. The exit channels 90 may each have a width of approximately 0.035 mm.

The arrangement and relative size of the exit channels 90 and fluid distributor channels 74, 75 are envisaged to at least reduce hydraulic stress on the local ocular tissue. It is understood that greater local supply (or ‘presentation pressure’) of aqueous humor may lead to a reduction in the absorption capacity of the tissue/capsule which forms around an implant. Accordingly, it will be appreciated that the exit channels 90 and the overall arrangement of the fluid network may at least promote preferential fluid distribution distally, away from the entry point to the ocular tissue, before releasing from the implant body 15. This is because, inter alia, the fluid distributor hub 80 in one embodiment is located distally along the implant body 15 (the hub 80 being at the site at which the fluid carrier channels 72 direct flow) and additionally or alternatively, due to the arrangement and relative size of the exit channels 90 and the fluid distributor channels 74, 75. Therefore, more fluid may exit the implant body 15 distally than anteriorly, and this specific flow distribution structure may at least match the relative capacity to absorb fluid (aqueous humor) for the ocular tissue at a distance. This specific flow distribution structure may also ensure that fluid is not distributed adjacent the anatomical limbus (i.e. near the entry point of the implant body 15, being the fluid port 25 of the frontal portion 20). This region is understood to have good absorptive capacity, but at higher levels of complications. It is envisaged that, in preferred embodiments, this specific flow distribution structure may ensure that fluid is not distributed to within approximately 3 mm of the anatomical limbus, and that a small proportion of the fluid drained from the eye is presented to tissues anterior to Tenon's insertion (anatomical). It will be appreciated that in embodiments, the majority of fluid drained from the eye will be presented to tissues between approximately 6 mm and 21 mm from the anatomical limbus, and preferably approximately 12 mm from the anatomical limbus.

The arrangement of the fluid network is also envisaged to provide microfluidic resistance to aqueous humour fluid flow of between 2.00 to 4.80 mmHg·min/uL, such that fluid flow from the implant body 15 may be optimised. In one form, the microfluidic resistance is between 3.80 to 4.00 mmHg·min/uL. In another form, the microfluidic resistance is between 2.80 to 3.80 mmHg·min/uL. In a preferred form, the microfluidic resistance is around 3.40 mmHg·min/uL. It is understood that too little fluid flow may result in the glaucoma not being effectively treated, whilst too much fluid flow may lead to hypotony, which is characterised as unhealthy low IOP. The microfluidic resistance may be calculated, for example, using the Hagen-Poiseuille formula (or derivations thereof) for fluid with dynamic viscosity of 0.72 mPa·s. The resistance may also be measured with or without the use of flow modification such as an intraluminal stent.

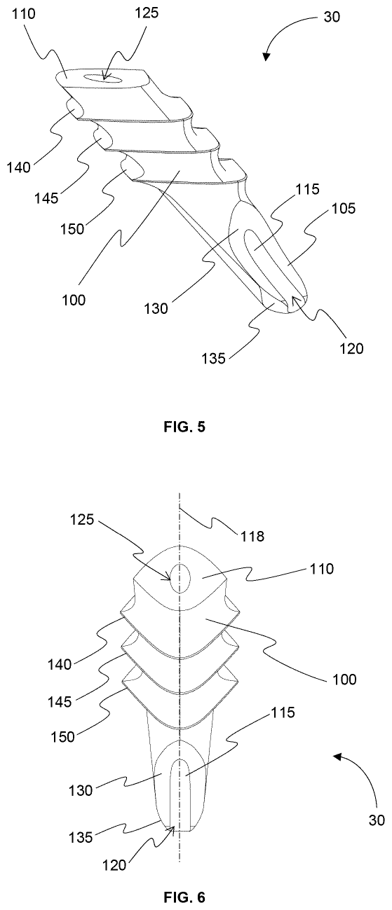

Returning to the fluid entry cannula 30 of the glaucoma device 10, and as best shown in FIGS. 5 to 8, the entry cannula 30 may include a cannula body 100 having a first end portion 105, a second end portion 110, and a lumen 115. The cannula body 100 is preferably formed from a flexible or elastomeric biocompatible material. A suitable biocompatible material may include, but is not limited to, a polymer material such as silicone or polyurethane. In one form, the material may be medical grade Polydimethylsiloxane (silicone), or PDMS. In a preferred form, the entry cannula 30 is separately articulated from the implant body 15. In embodiments (not shown), the entry cannula 30 may be integrally formed with the implant body 15. In certain embodiments, the entry cannula 30 may be provided as a separate device for various other applications, such as a diagnostic device or other device for drug delivery. As such, it will be appreciated that the entry cannula 30 may not necessarily be limited to applications involving glaucoma devices, and may include alternative or additional features that are customised for those other applications.

In one example of a diagnostic device application, the entry cannula 30 may be used during ophthalmic surgery to characterise the condition of the ocular tissue in patients with an elevated IOP and/or glaucoma, as a diagnostic aid. The entry cannula 30 may also be used to stabilise the IOP at safe levels (and prevent hypotony) in patients undergoing intraocular surgery.

It is envisaged that, in such diagnostic device applications, the entry cannula 30 may assist ophthalmic surgeons to understand the drainage capacity and permeability of the eye's trabecular network to predict the outflow functionality of the tissue's Effective Ocular Porosity (EOP).

In another example of a drug delivery application, the entry cannula 30 may be connected to a drug delivery reservoir device that allows slow release of a drug, substance or gene therapy vector to the intended tissues of a surgical site, for example. The surgical site may include the eye or alternatively non-ocular tissue. Ocular tissues may include the sub-retinal space, vitreous cavity or anterior chamber.

In an embodiment whereby the entry cannula 30 is used with a glaucoma device, the first end portion 105 of the entry cannula 30 may have a first opening 120 that is adapted for connection or insertion with the ocular tissue, and in particular, the anterior chamber of the eye. The second end portion 110 may have a second opening 125 that may be adapted for connection with the fluid port 25 of the frontal portion 20 of the implant body 15. In one form, the second portion 110 (the entry cannula ‘base’) may be wider than the first end portion 105 so as to allow for this second end portion 110 to have a strong connection or bond with the frontal portion 20 of the implant body 15. Additionally, this arrangement may allow for a tapered longitudinal profile of the cannula body 100 distally from the implant body 15 (i.e. smaller at the first end portion 105, larger at the second end portion 110) so as to allow for ease of implantation or insertion into the ocular tissue.

The lumen 115 extends between the first opening 120 and the second opening 125 so as to provide for fluid connection between the first end portion 105 and the second end portion 110. It is envisaged that the lumen 115 may open anteriorly and be angled away from the iris so as to reduce the likelihood the lumen 115 being occluded from iris blockage, as will be described in further detail below. In use, fluid from the ocular tissue may enter the entry cannula 30 via the first opening 120 and flow through the lumen 115 towards the second opening 125, following which, the fluid may enter the fluid port 25 of the frontal portion 20 of the implant body 15 to be dispersed therefrom.

In one form, the lumen 115 may have a diameter of at least 0.080 mm (80 micron) and in a preferred form, the lumen 115 has a diameter of approximately 0.127 mm (127 micron). It will be appreciated that the diameter of the lumen 115 may be large enough to have a low chance of occlusion by debris (e.g. debris present post-surgery, or particles or cells that are present in normal aqueous humor). It will, however, be appreciated that the dimensions of the lumen 115 are not necessarily limited to the preferred dimensions indicated above, and may be adjusted depending on the design requirements of the glaucoma device 10.

In the depicted embodiment, and as best shown in FIG. 5, the first end portion 105 may be provided with a bevelled or angled (removed) portion 130 and a rounded portion 135 to facilitate insertion into the ocular tissue. As discussed above, the bevelled or angled (removed) portion 130 may at least reduce the likelihood the lumen 115 being occluded from iris blockage. In a preferred form, the first end portion 105 may have a diameter that is less than 0.5 mm, and may be arranged such that it is unlikely to protrude more than 1 mm inside the anterior chamber of the eye. In one form, the rounded portion 135 may have a diameter of approximately 0.295 mm. In another form, the rounded portion 135 may have a diameter of approximately 0.200 mm. In a preferred form, the rounded portion 135 may have a diameter of approximately 0.206 mm. It will again be appreciated that the dimensions of the first end portion 105 are not necessarily limited to the preferred dimensions indicated above, and may be adjusted depending on the design requirements of the glaucoma device 10.

It will be appreciated that the overall shape and scale/dimensions of the cannula body 100 may match the track of a single or multi-bevel needle, such as a 27-gauge needle, for optimal insertion into the ocular tissue. Further, and as best shown in FIGS. 6 and 8, the canula body 100 may have an ellipse-shaped cross-section to facilitate fluid sealing with the entry hole of the ocular tissue. The two widest points of the ellipse-shaped cross section may also have a sharpened or pointed configuration. The ellipse-shaped cross-section of the cannula body 100 is envisaged to closely match the cross-section of the track or path created by the single or multi-bevel needle through the ocular tissue. The cross-section of the cannula body 100 may become more circular distally (i.e. toward the first end portion 105) to aid with ease of implantation.

In use, the entry cannula 30 may enter the ocular tissue such that it is angled at approximately 145 degrees anteriorly (relative to the implant body 15) to match the eye anatomy so as to reduce risk of occlusion and to improve ease of implantation. The overall length of the cannula body 100 may be between approximately 1.50 mm to 2.50 mm to penetrate through the cornea. In one form, the cannula body 100 may have an overall length of approximately 1.60 mm. In another form, the cannula body 100 may have an overall length of approximately 2.00 mm. The cannula body 100 may also have a width of between approximately 0.55 mm to 0.85 mm and a thickness/depth of between approximately 0.25 mm to 0.45 mm. In one form, the cannula body 100 may have a width of between approximately 0.59 mm to 0.75 mm and a thickness/depth of between approximately 0.28 mm to 0.40 mm. In another form, the cannula body 100 may have a width of between approximately 0.64 mm to 0.82 mm and a thickness/depth of between approximately 0.31 mm to 0.43 mm. It will again be appreciated that the dimensions of the cannula body 100 are not necessarily limited to the preferred dimensions indicated above, and may be adjusted depending on the design requirements of the glaucoma device 10.

The entry cannula 30 may include one or more ridges or flanges which project outwardly from the cannula body 100 to facilitate fixation/location of the entry cannula 30 in the ocular tissue and to reduce or prevent aqueous humor leakage. Each flange may be arranged to be parallel to the scleral tissue layers, and parallel to the implant body 15 in use. Each flange may also have a flared configuration, and may have a constant cross-section along the length of the cannula body 100. Each flange may project outwardly from the cannula body 100 in a direction transverse to a central longitudinal axis 118 of the lumen 115 or alternatively, transverse to the major direction of extension of the cannula body 100. Each flange may also have sharp edges and may be scooped backwards to seal the fluid and to allow for fixation of the entry cannula 30 within the surrounding layered eye tissue. It will be appreciated that the arrangement of the one or more flanges may at least prevent or reduce extra-luminal fluid flow between the outside of the entry cannula 30 and the ocular tissue and, as noted above, may reduce or prevent overall aqueous humor leakage. Accordingly, with this arrangement, the implantation of the glaucoma device 10 may be less likely to cause hypotony (low IOP) after surgery.

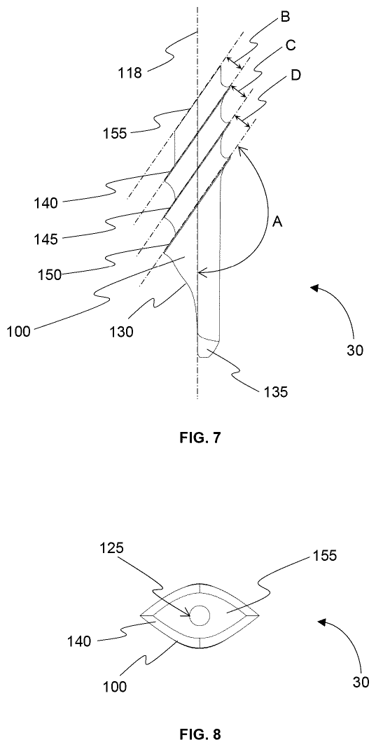

In particular, and as best shown in FIGS. 5 to 7, the cannula body 100 may include a first or base flange 140, and second or mid flange 145, and a third or end flange 150. Referring to FIG. 7, each flange may project at an angle A relative to the central longitudinal axis 118 of the lumen 115. The angle A may be between approximately 145 to 155 degrees. In one form, the angle A may be approximately 145 degrees. In another form, the angle A may be approximately 150 degrees. It will be appreciated that in this arrangement, a slightly flatter entry angle may at least allow for the tip of the implant body 15 to be located further from the limbus. The first or base flange 140 may have a distance B from a surface 155 of the second end portion 110. The second or mid flange 145 may have a distance C to the first or base flange 140. The third or end flange 150 may have a distance D to the second or mid flange 145. In one form, each distance B, C, and D may be 0.125 mm. In one form, each flange may also have a width of approximately 0.75 mm and a depth/thickness of approximately 0.4 mm. In another form, each flange may have a width of approximately 0.815 mm and a depth/thickness of approximately 0.422 mm. It will again be appreciated that the dimensions of the flanges are not necessarily limited to the preferred dimensions indicated above, and may be adjusted depending on the design requirements of the glaucoma device 10.

In FIGS. 9 to 12 of the accompanying drawings, there is schematically depicted a further embodiment of a glaucoma device 1010 and components thereof. It will be appreciated that the glaucoma device 1010 functions in a generally similar manner to the glaucoma device 10 described above, with like reference numerals being used to indicate like features. In the interest of clarity, the description of the like features of this embodiment will not be repeated, and it will be understood that any one or more of the like features or functions of the embodiment of the glaucoma device 10 described above are applicable to the embodiment of the glaucoma device 1010.

In this embodiment of the glaucoma device 1010, the implant body 1015 may have an overall length of approximately 19.5 mm, an overall width of approximately 5.5 mm, and an overall thickness/depth of approximately 0.21 mm. It is understood that that an implant body having a lower profile and width may at least reduce the risk of tissue erosion around the implant. In this embodiment, the thickness/depth may be constant.

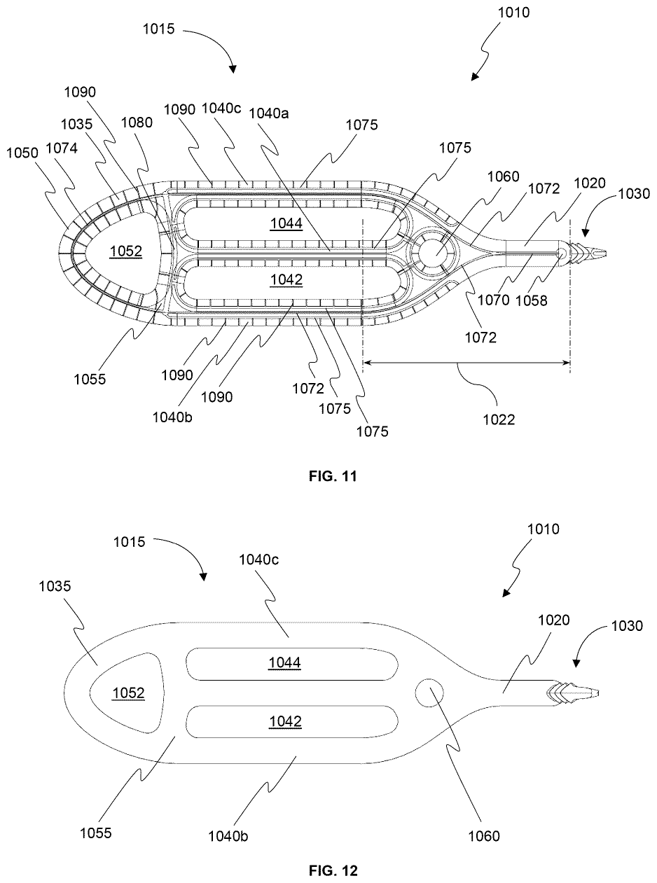

In the depicted embodiment, the stem or frontal (anterior) portion 1020 may have a length of approximately 2.50 mm, a width of approximately 1.00 mm and a low-profile thickness/depth of approximately 0.21 mm. It is understood that the height in this area of the implant body may be part of a mitigation strategy to reduce the risk of tissue erosion where the implant is the most exposed to overlying tissues. It is also anticipated that a reduced profile may lead to greater patient comfort. As indicated above, the overall width of the implant body 1015 may be approximately 5.5 mm. The implant body 1015 may have a shoulder portion 1022 (see FIG. 11), which may be defined as a distance between a straight part of the lateral arm portions 1040b, 1040c and the free end of the frontal portion 1020. This shoulder portion 1022 may have a length of approximately 8.00 mm. It is understood that this arrangement may at least reduce the risk of interference with the recti muscles adjacent to the implant.

In the depicted embodiment, the implant body 1015 includes a single suture opening or hole 1060. It is understood that this arrangement may at least allow for the narrowing of the overall width of the implant body 1015 (narrowing of the shoulder portion 1022), whilst retaining accessible suture holes. It is also understood that this arrangement may at least allow for improved manufacturability. In the embodiment shown, the suture hole 1060 has a circular shape, although it will be appreciated that the shape and configuration of the suture hole 1060 is not necessarily limited to the shape and configuration shown, and may be adjusted depending on the design requirements of the glaucoma device 1010.

As with the embodiment of the implant body 15 described above, the fluid network of the implant body 1015 may include a fluid network that extends from the frontal portion 1020, along at least one of the arm portions 1040, to the distal portion 1035. In this embodiment, the fluid network includes a fluid entry channel 1070 provided in the frontal portion 1020, a fluid carrier channel 1072 provided in each of the lateral arm portions 1040b, 1040c, and a distal fluid distributor channel 1074 provided in the distal portion 1035. As such, in this embodiment, the inner arm portion 1040a of the implant body 1015 does not include a fluid carrier channel. Each of the fluid carrier channels 1072 provided in the lateral arm portions 1040b, 1040c may have a width of approximately 0.068 mm. The fluid entry channel 1070 may have a width of approximately 0.120 mm and the fluid distributor channel 1074 may have a width of approximately 0.050 mm. The fluid network may also include an anterior fluid distributor channel 1075 provided in each of the lateral arm portions 1040b, 1040c. The fluid network may have an overall channel height of approximately 0.039 mm. As best shown in FIG. 11, fluid from the ocular tissue may travel along the fluid entry channel 1070, along each of the fluid carrier channels 1072, along the distal fluid distributor channel 1074, and along the fluid distributor hub 1080. The fluid may also loop back over the arm portions 1040 via the anterior fluid distributor channels 1075.

In this embodiment of the implant body 1015, the fluid network includes between 160 and 170 of the exit channels 1090. In a preferred form, the fluid network includes 163 of the exit channels 1090. The exit channels 1090 may each have a width of approximately 0.030 mm. In this embodiment, the suture hole 1060 is also provided with a plurality of the exit channels 1090 connected to a fluid distributor channel 1075 that surrounds the suture hole 1060. The arrangement of the fluid network is also envisaged to provide microfluidic resistance to aqueous humour fluid flow of between 2.80 to 3.80 mmHg. min/uL and preferably 3.20 to 3.40 mmHg. min/uL to allow for optimisation of post-operative eye pressures. It will also be appreciated that this arrangement of the fluid network may provide for a suitable ratio of resistance between the exit channels 1090 to the distributor channels 1074 and 1075, thereby allowing for more even distribution of aqueous flow and an increased to the effective surface area of the implant for fluid outflow.

Various forms of the glaucoma device described above may have one or more of the following advantages. The implant body is designed to provide an optimum specific resistance against aqueous humor fluid flow by way of the arrangement of the fluid channels of the fluid network. The magnitude of perfusion exit channels and the function of preferential distribution of fluid flow may at least reduce stress on local tissue. The entry cannula design may provide a sealed entrance to the ocular tissue, and may also reduce the risk of hypotony, balanced with providing ease of implantation. The glaucoma device is provided sterile and easy to use for surgeons, and is also designed to not be easily displaced after implantation. The overall arrangement of the implant body and the entry cannula may thus quickly reduce IOP to within a target range to treat glaucoma, and also improve long-term treatment outcomes by at least reducing the severity of the tissue reaction and preserving tissue response.

It will further be appreciated that various design features of the entry cannula may reduce the probability of intraocular inflammation or damage to the corneal endothelium of the eye. Such design features include, for example, the small size of the entry cannula to remain in situ within the anterior chamber, the ab externo insertion technique, angulation away from cornea and iris, multiple flanges to reduce movement of the entry cannula within tissue, and the cross-sectional shape and flanges acting to reduce fluid leak exterior to the entry cannula. Such advantageous design features also include the tapered profile of the cannula body, the centrally facing extending opening, and flange around the external opening

Although specific embodiments of the invention are illustrated and described herein, it will be appreciated by those of ordinary skill in the art that a variety of alternative and/or equivalent implementations exist. It should be appreciated that the exemplary embodiment or exemplary embodiments are examples only and are not intended to limit the scope, applicability, or configuration in any way. Rather, the foregoing summary and detailed description will provide those skilled in the art with a convenient road map for implementing at least one exemplary embodiment, it being understood that various changes may be made in the function and arrangement of elements described in an exemplary embodiment without departing from the scope as set forth in the appended claims and their legal equivalents. Generally, this application is intended to cover any adaptations or variations of the specific embodiments discussed herein.

It will also be appreciated that in this document the terms “comprise”, “comprising”, “include”, “including”, “contain”, “containing”, “have”, “having”, and any variations thereof, are intended to be understood in an inclusive (i.e. non-exclusive) sense, such that the process, method, device, apparatus or system described herein is not limited to those features or parts or elements or steps recited but may include other elements, features, parts or steps not expressly listed or inherent to such process, method, article, or apparatus. Furthermore, the terms “a” and “an” used herein are intended to be understood as meaning one or more unless explicitly stated otherwise. Moreover, the terms “first”, “second”, etc. are used merely as labels, and are not intended to impose numerical requirements on or to establish a certain ranking of importance of their objects.

### List of Reference Numerals

10 Glaucoma device 130 Bevelled or angled portion
15 Implant body 135 Rounded portion
20 Frontal portion 140 First or base flange
25 Fluid port 145 Second or mid flange
30 Entry cannula 150 Third or end flange
35 Distal portion 1010 Glaucoma device
40 Arm portions 1015 Implant body
40a Inner arm portion 1020 Frontal portion
40b First lateral arm portion 1022 Shoulder portion
40c Second lateral arm portion 1035 Distal portion
42 First window 1040 Arm portions
44 Second window 1040a Inner arm portion
50 Arcuate member 1040 b First lateral arm portion
52 Third window 1040c Second lateral arm portion
55 Bridge portion 1042 First window
60 Suture hole 1044 Second window
70 Fluid entry channel 1050 Arcuate member
72 Fluid carrier channel 1052 Third window
74 Distal fluid distributor channel 1055 Bridge portion
75 Anterior fluid distributor channel 1060 Suture hole
80 Fluid distributor hub 1070 Fluid entry channel
90 Exit channels 1072 Fluid carrier channel
100 Cannula body 1074 Distal fluid distributor channel
105 First end portion 1075 Anterior fluid distributor channel
110 Second end portion 1080 Fluid distributor hub
115 Lumen 1090 Exit channels
118 Central longitudinal axis
120 First opening
125 Second opening

1. A glaucoma device comprising:
an implant body including:
a frontal portion;
a distal portion;
a plurality of arm portions extending between the frontal portion and the distal portion so as to provide a connection therebetween; and
a fluid network extending from the frontal portion, along at least one of the arm portions, and to the distal portion, wherein the fluid network is provided with a plurality of exit channels for egress of fluid therefrom; and
a fluid entry cannula disposed at the frontal portion of the implant body and adapted for fluid connection with ocular tissue.

2. The glaucoma device according to claim 1, wherein the fluid network includes:
a fluid entry channel provided in the frontal portion;
a fluid carrier channel provided in at least one of the arm portions to carry fluid distally; and
a distal fluid distributor channel provided in the distal portion and connected to the plurality of exit channels.

3. The glaucoma device according to claim 2, wherein the fluid network further includes an anterior fluid distributor channel in at least one of the arm portions to carry fluid anteriorly to the plurality of exit channels.

4. The glaucoma device according to claim 3, wherein the fluid network further includes:
a fluid distributor hub providing a fluid connection between the fluid carrier channels and the distal fluid distributor channel, and wherein the anterior fluid distributor channels extend from the fluid distributor hub.

5. The glaucoma device according to claim 3 or 4, wherein the fluid network extends from the fluid entry channel provided in the frontal portion, along each of the fluid carrier channels of the arm portions, towards the distal fluid distributor channel provided in the distal portion, and returning along each of the anterior fluid distributor channels of the arm portions.

6. The glaucoma device according to claim 5, wherein the plurality of exit channels are provided in each of the distal and anterior fluid distributor channels so as to allow for egress of fluid from one or more of the arm portions preferentially in the distal portion.

7. The glaucoma device according to any one of the preceding claims, wherein the fluid network is provided with between approximately 70 and 220 of the exit channels.

8. The glaucoma device according to any one of the preceding claims, wherein the fluid network is provided with between approximately 150 and 180 of the exit channels.

9. The glaucoma device according to any one of the preceding claims, wherein the fluid network is provided with approximately 163 of the exit channels.

10. The glaucoma device according to any one of the preceding claims, wherein the fluid network is adapted to provide a resistance to aqueous humour fluid flow of between 2.00 to 4.80 mmHg·min/uL (Hagen-Poiseuille flow with fluid dynamic viscosity of 0.72 mPa·s).

11. The glaucoma device according to claim 10, wherein the fluid network is adapted to provide a resistance to aqueous humour fluid flow of between 2.80 to 3.80 mmHg. min/uL

12. The glaucoma device according to claim 10 or 11, wherein the resistance is measured with or without the use of flow modification such as an intraluminal stent.

13. The glaucoma device according to any one of the preceding claims, wherein the entry cannula includes:
a first end portion having a first opening adapted for connection with the ocular tissue; and
a second end portion having a second opening connected to a fluid port of the frontal portion of the implant body; and
a lumen extending between the first and second openings to provide for fluid connection between the first and second end portions.

14. The glaucoma device according to any one of the preceding claims, wherein the entry cannula extends at an angle of approximately 150 degrees relative to the implant body.

15. The glaucoma device according to any one of the preceding claims, wherein the entry cannula is articulated separately from the implant body.

16. The glaucoma device according to any one of the preceding claims, wherein the plurality of arm portions include:
an inner arm portion;
a first lateral arm portion disposed on a first side of the inner arm portion and spaced therefrom to form a first window; and
a second lateral arm portion disposed on a second side of the inner arm portion and spaced therefrom to form a second window.

17. The glaucoma device according to claim 16, wherein the distal portion extends between the first lateral portion to the second lateral portion so as to form a third window between the plurality of arm portions and the distal portion.

18. The glaucoma device according to any one of the preceding claims, wherein the implant body has a length of between approximately 16 and 23 millimetres and a width of between approximately 5 and 8 millimetres.

19. The glaucoma device according to claim 18, wherein the implant body has a length of approximately 19.5 millimetres and a width of approximately 5.5 millimetres.

20. The glaucoma device according to any one of the preceding claims, wherein the implant body has a thickness of less than approximately 0.6 millimetres.

21. The glaucoma device according to claim 20, wherein the implant body has a thickness of approximately 0.21 millimetres.

22. The glaucoma device according to any one of the preceding claims, wherein the implant body further includes at least one suture hole for surgical fixation of the implant body to the ocular tissue.

23. The glaucoma device according to claim 22, wherein the at least one suture hole is provided adjacent the plurality of arm portions, and wherein the plurality of exit channels are provided in a fluid distributor channel surrounding the suture hole so as to allow for egress of fluid thereform.

24. The glaucoma device according to any one of the preceding claims, wherein each of the arm portions has a width of approximately 1 millimetre.

25. The glaucoma device according to any one of the preceding claims, wherein the implant body is formed from a flexible or elastomeric polymer material.

26. The glaucoma device according to claim 25, wherein the implant body is formed from a medical grade Polydimethylsiloxane (silicone).

27. A glaucoma device comprising:
an implant body including:
a frontal portion adapted for fluid connection with ocular tissue;
a distal portion;
a plurality of arm portions extending between the frontal portion and the distal portion so as to provide a connection therebetween; and
a fluid network to provide internal fluid resistance, whereby the fluid network extends from the frontal portion, along at least one of the arm portions, and to the distal portion, wherein the fluid network is provided with a plurality of exit channels for egress of fluid therefrom.

28. An entry cannula for a glaucoma device, the entry cannula having a cannula body including:
a first end portion having a first opening adapted for connection with ocular tissue; and
a second end portion having a second opening adapted for connection with an implant body of a glaucoma device; and
a lumen extending between the first and second openings to provide for fluid connection between the first and second end portions.

29. The entry cannula according to claim 28, wherein the second end portion of the cannula body is wider than the first end portion of the cannula body.

30. The entry cannula according to claim 28 or 29, wherein the first end portion has a bevelled or removed portion for the lumen to open anteriorly from the iris tissue.

31. The entry cannula according to any one of claims 28 to 30, wherein the first end portion has a rounded portion for ease of insertion into the ocular tissue.

32. The entry cannula according to any one of claims 28 to 31, wherein the lumen has a diameter of at least 0.08 millimetres.

33. The entry cannula according to claim 32, wherein the lumen has a diameter of approximately 0.127 millimetres.

34. The entry cannula according to any one of claims 28 to 33, wherein the cannula body has an ellipse-shaped cross-section.

35. The entry cannula according to any one of claims 28 to 34, wherein the cannula body has a length of approximately 2.00 millimetres.

36. The entry cannula according to any one of claims 28 to 35, wherein the cannula body has a width of between approximately 0.64 to 0.82 millimetres.

37. The entry cannula according to any one of claims 28 to 36, wherein the cannula body has a thickness or depth of between approximately 0.31 to 0.43 millimetres.

38. The entry cannula according to any one of claims 28 to 37, wherein the cannula body further includes one or more flanges that project outwardly from the cannula body, so as to enhance sealing properties of the cannula body.

39. The entry cannula according to claim 38, wherein each flange projects outwardly from the cannula body in a direction parallel to the implant body of the glaucoma device and parallel to the corneal tissue.

40. The entry cannula according to claim 39, wherein each flange projects outwardly from the cannula body at an angle of between approximately 145 and 155 degrees.

41. The entry cannula according to claim 40, wherein each flange projects outwardly from the cannula body at an angle of approximately 150 degrees.

42. The entry cannula according to any one of claims 38 to 41, wherein each flange has a flared configuration.

43. The entry cannula according to any one of claims 38 to 42, wherein each flange has a width of approximately 0.815 millimetres and a depth/thickness of approximately 0.422 millimetres.

44. The entry cannula according to any one of claims 38 to 43, wherein the one or more flanges include:
a first flange disposed adjacent and spaced apart from a surface of the second end portion of the cannula body;
a second flange spaced apart from the first flange; and
a third flange spaced apart from the second flange.

45. The entry cannula according to claim 44, wherein the first flange is spaced apart from the surface of the second end portion by a distance of approximately 0.125 millimetres.

46. The entry cannula according to claim 45, wherein the second flange is spaced apart from the first flange by a distance of approximately 0.125 millimetres, and the third flange is spaced apart from the second flange by a distance of approximately 0.125 millimetres.
